(19) United States
(12) Patent Application Publication
(10) Pub. No.: US 2020/0031690 A1
(43) Pub. Date: Jan. 30, 2020

(54) Mobile Rhamnolipid Contamination Response

(71) Applicant: Keith DeSanto, St Petersburg, FL (US)

(72) Inventor: Keith DeSanto, St Petersburg, FL (US)

(21) Appl. No.: 16/513,292

(22) Filed: Jul. 16, 2019

Related U.S. Application Data
(60) Provisional application No. 62/702,513, filed on Jul. 24, 2018.

(51) Int. Cl. C02F 1/40; C07H 1/06; C07H 13/06; B09C 1/08

(52) U.S. Cl. CPC C02F 1/40; B09C 1/08; C07H 13/06; C07H 1/06

(57) ABSTRACT

Our mobile rhamnolipid production vehicles can convert pollutants obtained from contaminated sites into rhamnolipid. Then, our mobile rhamnolipid application units can alter, treat and remove pollutants from soil and water. Our new rhamnolipid application can use various sources for power. All these new components combined are cost efficient. Moreover, the mobile rhamnolipid contamination response vehicles are easy to mobilize and can be deployed and up and running with a short amount of time and effort.

Our mobile rhamnolipid treatment and application units can alter, treat and remove pollutants such as oil, diesel (hydrocarbons) and metals from soil and water. Our mobile Rhamnolipid production vehicles (some being powered by solar panels), can convert the hydrocarbon pollutants into rhamnolipid. The rhamnolipid can then be used to break apart the petroleum oil and diesel oil “hydrocarbons” in the water and soil. By using solar panels for power generation and by using the petroleum oil and diesel oil pollutant as a substrate to feed bacteria that produces rhamnolipid, the mobile units can be deployed quickly and with ease for cost effective environmental cleanup and treatment applications.

It is well known through various third-party publications that rhamnolipid(s) can sequester metals from soil and water where the rhamnolipid(s) bind to metals is stronger than that of the surrounding soil. It is also well known through various third-party publications that rhamnolipid(s) can break apart hydrocarbons making it easier for environmental contamination cleanup. Our new rhamnolipid mobile application uses pollutant substrates to make rhamnolipid at the site of environmental contamination. Our new rhamnolipid application can use either local electrical power or wind, solar or flowing water generated power. All these new applications and state of the art communication sources combined are cost efficient and easy to mobilize. They can be deployed and up and running with a short amount of time and effort and can use various sources for power.

1. An application where, rhamnolipid production tanks for producing rhamnolipid are fixed on a vehicle that is mobile.

2. An application as in claim 1 where the mobile vehicle can be a boat, ship, train, jet, airplane or land roaming vehicle.

3. An application as in claims 1 and 2 where each rhamnolipid production tank is short and stacked on top of one another.

4. An application as in claims 1, 2 and 3 where the rhamnolipid production tanks are stacked on top of one another and placed side by side and be made of plastic, metal or a combination of both.

5. An application in claim 1 where the rhamnolipid production tanks are connected to other similar mobile vehicle units.

6. An application as in claims 1 through 5 where the rhamnolipid production tanks are connected to each other by metal pipe and or plastic pipe and or electrical power sources.

7. An application as in claims 1 through 6 where the rhamnolipid production tanks are powered by solar power, natural gas, power from running water, wind turbines or electricity.

8. An application as in claims 1 through 7 where the rhamnolipid production tanks are connected and operated wirelessly.

9. An application as in claims 1 through 8 where the rhamnolipid production tanks are monitored and or managed by drones from land, from water or from the air.

10. An application as in claims 1 through 9 where the rhamnolipid production tanks are connected wirelessly by drones for communication purposes.

11. An application using claims 1 through 10 to remove oil and diesel from water.

12. An application using claims 1 through 11 to remove hydrocarbons such as oil and diesel from water and soil.

13. An application using claims 1 through 11 to sequester metals from water and soil.
